(19) United States
(12) Patent Application Publication
(10) Pub. No.: US 2020/0376154 A1
(43) Pub. Date: Dec. 3, 2020

(54) PROTEINS HAVING WOUND HEALING EFFICACY AND METHOD FOR ISOLATION FROM HUMAN HAIR

(71) Applicant: CELL CONSTRUCTS I, LLC, Marietta, GA (US)

(72) Inventors: Thomas BARROWS, Austell, GA (US); Nicholas BESKID, Atlanta, GA (US); Christopher INGRAHAM, Marietta, GA (US); Alexander MACDOUGALL, Atlanta, GA (US)

(21) Appl. No.: 16/971,466

(22) PCT Filed: Feb. 20, 2019
(86) PCT No.: PCT/US19/18696

§ 371 Date: Aug. 20, 2020

Related U.S. Application Data
(60) Provisional application No. 62/632,753, filed on Feb. 20, 2018.

(51) Int. Cl. A61L 15/32; A61L 15/22; A61L 15/28; C08H 1/06

(52) U.S. Cl. CPC A61L 15/32; A61L 15/225; C08L 5/02; C08H 1/06; A61L 15/28

(57) ABSTRACT

The invention is a non-hydrated, keratin wound dressing film comprising a fraction of keratins isolated from a solution of extracted keratins. Said fraction enables the formation of film with greater flexibility that can be achieved in film made from the unfractionated keratin extract. Also disclosed is a process of forming wound dressings and a method of obtaining said keratin fraction from an extract of human hair or other keratin raw materials.

### CROSS-REFERENCE TO RELATED APPLICATIONS

This application claims benefit of U.S. Provisional Application No. 62/632,753 filed Feb. 20, 2018, incorporated herein by reference in its entirety.

### TECHNICAL FIELD

The Technical Field includes biomaterials that accelerate wound healing and means for their efficient administration to facilitate optimal wound healing.

### BACKGROUND

Human skin, and the skin of all animals, protects the body from the external world of microorganisms within which higher life forms evolved. In fact, certain types of bacteria are ever present on skin and their numbers are controlled both by the constant self-renewal of skin and its composition that includes antimicrobial biomolecules. Thus, rapid healing of cutaneous wounds in concert with other mechanisms of defense against bacterial infection is essential for survival.

Most superficial wounds involving broken skin on healthy individuals heal rapidly without the need for intervention beyond keeping the wound protected and free of contamination. However, many conditions such as old age, diabetes, poor circulation of blood in the legs, and pressure ischemia as seen in bed-ridden invalids can delay or even prevent wound healing. Dealing with such chronic wounds continues to be a major unsolved medical problem causing untold human suffering and an enormous cost burden to the healthcare system.

The problem of delayed wound healing ideally could be solved with a wound dressing that interacts with the wound bed to promote healing by actively modifying the wound environment to compensate for deficiencies manifest in a delayed-healing wound. Yet beyond the obvious need to control infection and provide adequate moisture, there is no consensus on which of the multitude of potentially harmful conditions contributing to wound healing delay are amenable to being solved via dressing composition. Nevertheless, a plethora of commercially available dressings purport to improve wound healing by various hypothetical means, especially in the case of human amniotic membrane wound dressings that contain detectable amounts of growth factors and cytokines.

The use of human cadaver skin to cover third-degree burns is a well-accepted practice. Additionally, porcine dermis and porcine intestinal submucosa have been processed into wound covering products. The “bio-inspired” approach to wound care product development also has led to utilization of other natural membranes including amphibian skin, fish skin, and internal membranes such as horse pericardium. Although it might seem fruitful to extract the active agents from these materials, such as antimicrobial peptides and/or factors that accelerate healing, such efforts have not produced therapeutics in useful yield or in possession of adequate potency or stability. An alternative approach has been to incorporate crude membrane extracts or particles into other dressing materials to imbue synthetic dressings with the attributes of whole biological membranes. A common example is the combination of porcine dermal collagen with synthetic polymers to produce a variety of so-called “collagen” wound dressing.

In recent years, human proteins extracted from hair clippings (an unregulated waste material) have become recognized as having biological activity important for wound healing. Because keratins are a category of proteins that emerged at an ancient time of evolution, it is likely that keratins from other sources including wool, feathers, and hoofs might provide suitable substitutes for human hair as a raw material. An example of a human hair-derived wound treatment product is ProgenaMatrix™ (Cell Constructs I, LLC, Marietta, Ga. 30067), a clear, highly hydrated matrix that is purely comprised of reconstituted, non-oxidized human proteins without the use of additives or chemical crosslinking agents. In a diabetic mouse model of delayed healing, ProgenaMatrix™ was discovered to possess wound-healing efficacy on par with allograft human tissues. In subsequent experiments it was further discovered that the observed ProgenaMatrix™-induced accelerated wound closure appears to be due to an initial interaction of the keratin with the wound bed that preserves cell viability and enables proliferation of pro-regenerative macrophages, which likely contribute to a shortened inflammatory phase of wound healing leading to faster wound closure.

There exists a need in the art for a soft, pliable, drapable keratin dressing in the form of a thin film that is not highly hydrated and does not need to be packaged wet. It would also be useful to have a dry, yet flexible keratin film comprised of additional substances such as particles of animal or human skin and other membranes or biomolecules of known wound healing utility. It would further be desirable to be able to incorporate additives of natural or synthetic composition for the purpose of allowing the dry film to hydrate with an accelerated rate such that dressing changes necessitating removal and replacement of the film could be facilitated by simply rinsing off extant disintegrated keratin film residue during the dressing change procedure.

Optionally, in some instances it would also be desirable to incorporate antimicrobial agents such as silver and various silver compounds found in dressings that are commonly applied on infected or previously infected wounds. It is becoming increasing apparent, however, that harshly antiseptic substance including silver salts and iodine must be used sparingly so that their harmful effects on wound healing don't exceed their benefit of suppressing infection. Other anti-infection agents that could be added include, but are not limited to, bismuth tribromophenate, chlorhexidine, and polyhexamethylene biguanide (PHMB).

Separating, isolating, and identifying individual proteins obscured within complex mixtures is an important branch of biochemistry that relies upon various chromatographic techniques and, more recently, proteomic methods of analysis. In the case of keratin proteins, analysis is made difficult by the presence of intermolecular disulfide crosslinks that render the keratins insoluble. Since chromatography requires the proteins to be fully dissolved, disulfide bridges linking protein molecules together via their cysteine amino acid residues must first be broken to liberate single strands of polymeric molecules. This is routinely achieved using a reducing agent to convert the disulfide linkages into separated sulfhydryl residues (i.e. —S—S— +H₂= —SH+HS—).

To facilitate extensive processing without the problem of spontaneous crosslink reformation, separation of the disulfide bridges is typically made permanent by alkylation with iodoacetic acid or iodoacetamide (i.e. —SH+I—CH₂—COOH= —S—CH₂—COOH+HI) or by oxidation of the disulfide bridges into separate sulfonic acid moieties (i.e. —S—S— +3 O₂=2 —SO₃⁻). Oxidized keratin is known as “keratose”, a hydrophilic substance that has been utilized in the manufacture of wound care products derived from both sheep wool (Keramatrix, Keraplast Research, Templeton, New Zealand) and human hair (KeraStat® Gel, KeraNetics, LLC, Winston-Salem, N.C. 27101).

Unfortunately, such derivatization is not satisfactory as a manufacturing process for human hair-derived wound treatment products such as ProgenaMatrix™ because the resultant proteins, though amenable to traditional separation means, lack the capacity for disulfide bond reformation, which is critically important in the development of mechanical strength during the film formation process. Moreover, traditional protein fractionation methods having the highest resolving power cannot be scaled-up economically to enable commercial production except in the case of very high value parenteral drugs.

The principal constituent of human hair is a mixture of keratin and keratin-associated proteins. The hair-specific keratins are divided almost equally into type I acidic keratins (e.g. K31-38), and type II neutral/basic keratins (e.g. K81-86) that exist together as heterodimeric pairs. The keratin-associated proteins are numerous with more than 100 having been identified. The type II hair keratins are described as “hard” and are found to be required for proper hair cortex formation. Defective biosynthesis of type II keratins results in monilethrix, a rare inherited disorder characterized by sparse, dry, and/or brittle hair that often breaks before reaching more than a few inches in length.

An aspect of the present invention is that it provides an efficient and low-cost means of fractionating mixtures of keratin proteins extracted from human or animal keratin raw materials to isolate fractions having improved attributes relative to product performance while preserving or enhancing wound healing efficacy.

A further aspect of the invention is that it provides a means of converting said fractionated material into soft, flexible wound dressing films that can be packaged dry and may also incorporate other substances that modify its physical attributes, wound healing efficacy, or antimicrobial properties.

### SUMMARY

Disclosed herein is a film comprising a fraction of keratin proteins separated from an original extracted keratin protein mixture, wherein said film comprises consolidated particles of said fraction; wherein said film possesses a greater flex-fatigue life and/or exhibits a greater drape angle than a comparable film comprising consolidated particles of the original extracted keratin protein mixture from which it was fractionally separated. Said original extracted keratin protein mixture can be obtained from human hair and said fraction of keratin protein comprises a lower content of the keratins KRT85 and KRT86 relative to said original extracted keratin protein mixture. Said extracted keratin proteins can be obtained from feathers.

Also disclosed is the process of obtaining a fraction of keratin proteins by: a) providing a solution of keratin proteins by extracting a keratin raw material in an alkaline aqueous solution comprised of a reducing agent, a denaturing agent, and buffer salts; b) fractionally precipitating keratins from the solution of keratin proteins of step a) by addition of a first water-miscible organic solvent and collecting the solution of the remaining non-precipitated keratins by filtration; c) further fractionally precipitating keratins from the resultant solution of step b) by addition of a second water-miscible organic solvent and collecting the precipitated fraction of keratin solids on a filter; thereby obtaining said fraction of keratin proteins. Said first water-miscible organic solvent can be ethanol and said second water-miscible organic solvent can be acetone.

Further disclosed is a process of obtaining a fraction of keratin proteins comprising: a) providing a keratin material in granular form; b) sequentially extracting the keratin granules of step a) with a series of eluents having a constant concentration of denaturing agent and pH, but containing an increasing concentration of reducing agent; c) separately precipitating the keratin content of each eluent in a water-miscible organic solvent and collecting each precipitate on a filter; d) selecting the precipitate fraction desired; thereby obtaining a fraction of keratin proteins.

The keratin material of step a can be a recycled keratin material.

Also disclosed is a process of obtaining a film, wherein said processing comprises the steps of: a) providing a fraction of keratin proteins comprising precipitated keratin according to the process describe above; b) adding water to the precipitated keratin of step a and mixing to produce a slurry of particles; c) placing the slurry of step b) into a mold; d) allowing the water to evaporate; and e) removing the resultant film from the mold. Disclosed is a film made by this process.

The film disclosed herein can comprise substances beneficial to wound healing selected from the group comprising human and animal tissues and membranes and substances derived from said tissues and membranes. Said added substances can form a layer on one side of the film. Said tissues can be selected from the group comprising amniotic membrane, epidermis, and corneal epithelium. The film can comprise a hydroactive polymer. Said hydroactive polymer can be selected from the group comprising pullulan, pluronic, CMC, alginate, and dextran.

The film disclosed herein can comprise a fraction of keratin proteins separated from an original extracted keratin protein mixture can withstand at least 2 more cycles of flexing compared to the original extracted keratin protein mixture from which it is extracted.

The film can comprise a fraction of keratin proteins separated from an original extracted keratin protein mixture comprises 10° or greater drape angle compared to the original extracted keratin protein mixture from which it is extracted.

The fraction of keratin proteins separated from the original extracted keratin protein mixture in the film disclosed herein can comprise 80% to 90% of the proteins in the originally extracted keratin material.

Further, disclosed herein is a method of treating a subject, the method comprising administering the film of claim 1 to a subject in need thereof.

### BRIEF DESCRIPTION OF THE FIGURES

FIG. 1 is a graphical representation of the relative percentages of major human hair keratin subtypes determined by proteomic analysis to be present in film made from fractionally precipitated HPE in comparison to film make from non-fractionally precipitated HPE.

FIG. 2 is a graphical representation of the flex-fatigue life test results for films made from fractionated HPE (F2 keratin) vs. non-fractionated HPE (WP keratin).

FIG. 3 is a graphical representation of the drape angle measurement test results for films made from fractionated vs. non-fractionated HPE.

FIG. 4a is a photograph of the feathers used to prepare the fractionated keratin film of Example 5.

FIG. 4b is a photograph of the fractionated feather keratin film of Example 5.

FIG. 5 is a graphical representation of the percent yield of recovered protein obtained by sequential extraction of dried ProgenaMatrix™ granules vs. the BME content of the eluents utilized in each step of the extraction process.

FIG. 6a is a photograph of the extract precipitate of Example 7 obtain upon mixing a “fraction no. 2” extract with denatured alcohol and allowing it to coagulate.

FIG. 6b is a photograph of the precipitate of FIG. 6a being collected in a stainless-steel strainer.

FIG. 6c is a photograph of the precipitate of FIG. 6b after it was stirred with a 1:1 mixture of deionized water and denatured alcohol to remove soluble chemicals and subsequently collected on filter paper.

FIG. 6d is a photograph of the ProgenaMatrix™ granules collected on a filter after having been extracted with the eluent that produced fraction no. 2.

FIG. 7 is a photograph of the keratin-amniotic membrane film of Example 9.

FIG. 8a is a photograph of the porcine epidermis prepared by the process of Example 10 immediately prior to being combine with F2 slurry.

FIG. 8b is a photograph of the keratin-porcine epidermis film of Example 10.

FIG. 9a is a photograph showing corneal epithelium being removed from the stroma of a porcine cornea with the use of a hemostat.

FIG. 9b is a photograph of porcine corneal epithelium after having been homogenized in ethyl alcohol with the use of a motor-driven rotor-stator tissue homogenizer.

FIG. 9c is a photograph of the epithelial tissue particles of FIG. 9b after having been collected on a nylon net filter.

FIG. 9d is a photograph of the film that resulted from drying of the particles shown in FIG. 9c.

FIG. 9e is a photograph of the film that was produced by casting a slurry of F2 keratin of Example 1 onto the film of FIG. 9d.

FIG. 9f is a photograph of the portion of the film of FIG. 9e showing the transparent region of the film that is a bilayer composite of fractionated human keratin and porcine corneal epithelium.

FIG. 10 is a sketch of the diabetic foot ulcer model (1) of Example 14 in which the wound cavity (2) is fitted with a wound fluid inlet hypodermic needle (3) attached to tubing (4) that is connected to a syringe pump (not shown). Prior to admitting wound fluid into the cavity to simulate an exuding wound, the test material (5) is placed into the wound and covered with Adaptic® non-adherent dressing (6) and two layers of gauze (7, only one layer shown). The entire foot and dressings are then secured in place with Coban™ compression wrap (not shown).

FIG. 11 is a graph of the average weekly wound size measurements recorded during the healing of wounds created in diabetic mice treated with and without the wound dressing film of Example 12 showing faster wound healing in the treated mice.

### GENERAL DESCRIPTION

### Definition of Terms

The following terms as used herein shall have these defined meanings:

“BME” means beta-mercaptoethanol, also known as 2-mercaptoethanol.

“CMC” means carboxymethyl cellulose.

“Denaturing agent” means a water-soluble chemical compound that causes protein molecules to unfold, dissociate, or otherwise change their conformation in such a way as to become more water soluble and/or extractable from solid phase substances. Examples include but are not limited to urea and thiourea.

“Drape angle” means the magnitude of drape measured as follows: A one-centimeter wide strip of the film to be tested is advanced over the edge of a platform until the leading edge of the unsupported film is one centimeter beyond the platform. The drape angle of the film is measured as the angle formed between the leading edge of the film and the horizontal of the platform. Thus, with no drape the drape angle would be zero degrees (0°) and with 100% drape the drape angle wound be ninety degrees (90°). Drape is dependent on the thickness of the film, which must be measured and reported as a condition of drape angle determination. Ambient temperature and humidity also must be recorded during acquisition of drape angle measurements. The comparison of the drape of two or more different materials in film-form requires comparable sample thicknesses and similar conditions of ambient temperature and humidity during the time that the measurements are obtained.

“Drape” means the characteristic of a film that makes it feel limp rather than stiff.

“Eluent” means an aqueous solution having a defined composition comprising a specific concentration of reducing agent suitable to solubilize and extract a fraction of the keratin molecules contained in an insoluble keratin-containing substance.

“FPE” means feather protein extract.

“Flex-fatigue life” (“FFL”) means the characteristic of a film to withstand repeated cyclical loads of bending stress without cracking or breaking and is measured as follows: A one-centimeter wide strip of the film to be tested is placed over the edge of a platform and the supported portion is clamped in place to prevent movement. The portion of the film projecting over the edge is then manually flexed downward to form a 90° angle with respect to the surface of the platform. At this point the test begins and the film is bent upward 180° to form a new 90° angle with respect to the surface of the platform. In the second flex cycle and each flex cycle thereafter, the film is bend downward 180° (flex count number 2) and then upward 180° (flex count number 3) until 100 cycle of flex have been counted or until the sample cracks or breaks. The number of such bending cycles required to produce a defect or to break the film is recorded and reported as the FFL of the sample. Flex-fatigue life is dependent on the thickness of the film, which must be measured and reported as a condition of FFL determination. Ambient temperature and humidity also must be recorded during acquisition of FFL measurements. The comparison of the FFL of two or more different materials in film-form requires comparable sample thicknesses and similar conditions of ambient temperature and humidity during the time that the measurements are obtained.

“Flexibility” means a property of a film that allows it to bend without breaking. In comparing two films, the one with greater flexibility will have both a higher drape angle and greater flex-fatigue life.

“HPE” means human hair protein extract.

“HPEC” means human hair protein extract concentrate, which is HPE that has been concentrated by ultrafiltration.

“Hydroactive” material means a substance such as polymer, gel, or mixture of substances having high affinity for water that is either water soluble or water absorptive, but in its hydroactive form is substantially devoid of water.

“Hydrogel” means any non-liquid substance that is uniformly comprised of greater than about 40% water.

“KAP” means keratin associated protein (also abbreviated as “KRTAP”).

“Keratin wound dressing” means any product or experimental composition containing keratin that is intended for use in treating a wound to facilitate wound healing.

“Keratin” (also abbreviated as “KRT” or simply “K”) means any substance obtained from human hair or animal sources including, but not limited to, wool, hair, hooves, fur, and feathers; no matter how crude, refined, processed, fractionated, purified, comminuted, chemically derivatized, blended, copolymerized, or otherwise changed or altered in any way by any means. It also encompasses mixtures of keratin with KAPs and mixtures of keratin with non-keratin substances that are not readily separable from keratin by practical means.

“Keratin raw material” means a keratin-containing substance that has not previously been processed in preparation for manufacturing a keratin-containing product.

“MWCO” means molecular weight cut-off, a specification of ultrafiltration membranes indicating that molecules have a lower molecular weight than the specified number will pass through the membrane and that molecules having a higher molecular weight will not.

“Pluronic” means a class of hydrophilic non-ionic surfactants also known as Poloxamer surfactants that are block co-polymers of polypropylene glycol (“PPG”) and polyethylene glycol (“PEG”), wherein the hydrophobic PPG block comprises a central block flanked by two hydrophilic PEG end-blocks. Pluronic F-127, also known as Poloxamer 407, is a specific example of a pluronic that is widely used in medical and pharmaceutical applications.

“Recycled keratin material” means any keratin-containing substance that may become available as scraps, surplus, byproduct, or repurposed supplies that have been subjected to at least a minimal degree of processing. Recycled keratin materials include unused, extant, expired, or recovered keratin wound dressings.

“Reducing agent” means any water-soluble chemical compound that has the capacity to separate disulfide bonds (e.g. cystine) into dissociated sulfhydryl moieties (e.g. cysteine). Examples include, but are not limited to BME, dithioerythritol, dithiothreitol, dithiobutylamine, cysteamine, sodium sulfide, sodium bisulfite, tris(2-carboxyethyl)phosphine hydrochloride, and related phosphines and sulfides.

“SDS” means sodium dodecylsulfate, a detergent commonly used to decellularize tissue.

“Shindai” method or process means the conditions and methods utilized to extract keratin proteins from a keratin raw material comprising heating the keratin raw material at about 50° C. for about 2-4 days in a solution nominally buffered at pH 8.5 and containing approximately 5M urea, 2.5M thiourea, and 5% BME.

“Tris” or “tris base” means tris(hydroxymethyl)aminomethane. “Tris acid” is the hydrochloride salt of tris.

“Wound” means a surgical incision, excision, traumatic injury, burn, or ulcer in which skin is missing at least its entire epidermal layer and at most all its dermis and a substantial amount of subdermal flesh.

### DETAILED DESCRIPTION

The present invention is a wound dressing film comprised of keratin proteins having a ratio of keratin subtypes significantly altered from the ratio of keratin subtypes naturally present in the keratin raw material from which the wound dressing keratins are obtained, resulting in a wound dressing film with greater flexibility. It is also a method of altering the ratio of keratin subtypes by means of fractional precipitation of a solution of keratin proteins or, alternatively, fractional extraction of keratin subtypes from a keratin composition in solid form.

In one embodiment, the keratin raw material is human hair clippings solubilized by the Shindai method and the fractional precipitation is accomplished by pouring the resultant HPE into ethyl alcohol with vigorous mixing to precipitate a portion of the keratins present in the HPE solution. The solids resulting from that first precipitation step are collected by filtration and discarded. The keratins that remain soluble in the solution that now is a mixture of ethyl alcohol and Shindai solution is further precipitated in acetone and those precipitated solids are collected by filtration and saved for use in making the wound dressing film. Other keratin raw materials such as feathers that can be solubilized or partially solubilized by the Shindai method or similar means may also be processed by this method.

In another embodiment, the keratin raw material is dried scraps in granular form of a keratin wound dressing material provided as a hydrogel film comprising denatured human hair biomolecules intermolecularly crosslinked with disulfide bonds. An example of such a wound dressing material that can be converted into dry, granular form is disclosed in U.S. Pat. No. 9,072,818, but also could be keratin granules of any other solid keratin raw or recycled material. The granules are sequentially extracted with eluants having increasing power to reduce (i.e. reversibly break) disulfide bridges. Although 2-mercaptoethanol is a preferred reducing agent, numerous alternative chemicals may be used.

Thus, keratin granules are initially stirred in a first eluent solution having very low reducing agent concentration. The undissolved granules are collected on a filter and the dissolved keratin present in the filtrate precipitated by mixing with a water-soluble organic solvent such as ethyl alcohol. The collected granules are then stirred with a second eluent solution having a higher reducing agent concentration than the first eluent and the process repeated to precipitate a second keratin fraction. This process is repeated with increasing reducing agent concentration until virtually all the original keratin granules have been dissolved and recovered as a series of precipitated keratin fractions or until the residual undissolved keratin becomes too gelatinous to allow further processing by this method.

Unlike prior art methods of protein separation, the above methods of fractionating keratin proteins is independent of the molecular weight of the molecules being separated. It is also independent of their isoelectric points, and independent of their hydrophobicity or hydrophilicity. Thus, the molecules are only solubilized and separated into fractions based upon the density of disulfide crosslinks preventing solubilization at each specific reducing agent concentration in the case of the fractional extraction method and only upon their ability to remain solubilized in a specific aqueous/organic solvent mixture composition in the case of the fractional precipitation method.

Of course, any fraction thus collected may then be subjected to further fractionation by selecting another set extraction or precipitation solutions having a narrower range of incremental reducing agent concentration or solvation strength. Additional fractionation optionally may also include traditional methods such as density gradient centrifugation, ultrafiltration utilizing molecular weight range specific (MWCO) membranes, electrophoretic migration, ion-exchange chromatography, and affinity chromatography. Other potentially useful modifications to the fractionation and precipitation methods claimed herein will be apparent to those skilled in the art of protein separation technologies.

In the case where a precipitated fraction of keratin obtained by means described herein is to be re-eluted with a second series of eluents, it was found that the process is best facilitated by converting the precipitate back into a granular form. This can be accomplished by rinsing the collected precipitate with pure water and allowing it to dry into a solid mass. The solid mass can then be ground into granules for further extraction.

Each such isolated fraction has the potential of exhibiting a varying degree of wound healing efficacy upon conversion into a suitable wound dressing being evaluated in a relevant animal model of delayed wound healing. Although a primary objective of the invention is to provide a keratin wound dressing in the form of a dry, flexible film, it is envisioned that the fraction affording the greatest flexibility may also be supplemented with a fraction affording less flexibility but possessing greater wound healing efficacy.

In one example, the starting material can comprise a keratin wound dressing, such as the one disclosed in U.S. Pat. No. 9,072,818 (incorporated by reference herein for its teaching concerning keratin wound dressings). Disclosed herein is a process that begins with human hair being extracted with a solution of urea, thiourea, and 2-mercaptoethanol at pH 8.5 for 3 days at 50° C. (the “Shindai” process). The resultant HPE is then concentrated by ultrafiltration using a 30,000 Dalton MWCO membrane to produce HPEC that is subsequently converted directly into a clear, solid matrix comprising about 70% water. Thus, the hydrogel matrix itself is produced from a fractionated material. About 36% of the starting human hair becomes solubilized in making HPE; the residual undissolved hair having been discarded. Then, during concentration of HPE via ultrafiltration, about 10% of the proteins present in HPE are discarded because they pass through the 30,000 Dalton MWCO filter.

A finishing step in the manufacturing of the hydrogel matrix described above involves trimming the edges of large sheets prior to cutting them into specific sizes to be packaged and sterilized. The scraps from trimming are a useful starting material for fractionation. Scraps are rinsed with distilled water and allowed to dry in open air for several days until brittle; then ground into granules. The granules are then processed as discussed above to yield precipitated keratin fractions. It was discovered that a less concentrated solution of BME was needed to solubilize the hydrogel matrix granules than called for in the Shindai process. Thus, relative to Shindai conditions, eluents had half the concentration of urea and thiourea (same pH) and less than half the BME concentration.

In the case of the fractional precipitation method disclosed herein, a convenient starting material exists in the form of the same HPE required in the production of the hydrogel matrix described in U.S. Pat. No. 9,072,818. Although the Shindai process was specifically optimized for recovering keratins from human hair, it also serves as a useful means of obtaining keratins from other sources. One such source of keratin that presently has no commercial value and in fact creates a waste disposal problem is feathers from the poultry industry. Previous attempts at recovering keratin from feathers to produce textile fibers and other useful products has not yet achieved commercial success. However, in the case of biomaterials research it appears that feather keratin has the same cytocompatibility attributes as keratin from mammalian sources.

An additional embodiment of the present invention is a simple and efficient means of converting a precipitated keratin fraction as described above into a film. Accordingly, the precipitated keratin fraction is rinsed with a mixture of alcohol and water, collected on a filter, and then resuspended as a slurry in water without any intermediate drying and poured onto a flat surface, for example a silicone rubber sheet having a surrounding ring or lip to contain the pool of slurry and allowing the water to evaporate. As the water evaporates the fine particles of suspended keratin coalesce into a continuous layer that cures into film that can be peeled off the surface of the mold. The thickness of the film is dependent on the percent solids in the slurry and the volume of slurry deposited within the surface area defined by the mold.

As illustrated in Example 4, a film made from keratin subjected to the above fractional precipitation in comparison to a film made from the same keratin not subjected to the above fractional precipitation exhibited substantially greater flex-fatigue life. Therefore, the film made from keratin subjected to the above fractional precipitation in comparison to a film made from the same keratin not subjected to the above fractional precipitation can withstand 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82 82, 83, 84, 85, 86, 87, 88, 89, 90, 91, 92, 93, 94, 95, 96, 97, 98, 99, or 100 or more FFL bend cycles compared to the original film.

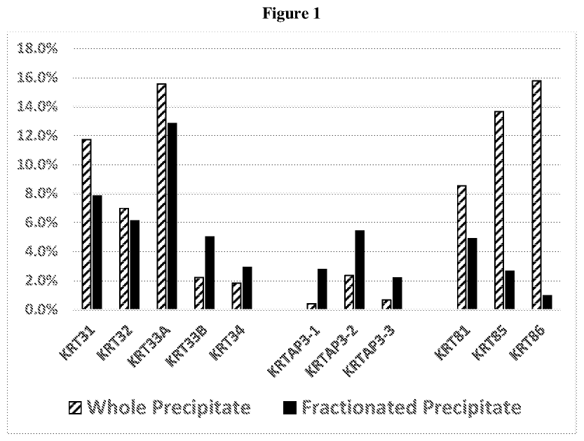

Moreover, proteomic analysis of these two keratin preparations revealed a significant alteration in the ratio of keratin subtypes and most notably a reduction in the content of K-85 and K-86, known as hard keratins that logically would be responsible for imparting stiffness to the film. These finding are presented in FIG. 1.

The film made from keratin subjected to the above fractional precipitation can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82 82, 83, 84, 85, 86, 87, 88, 89, 90, 91, 92, 93, 94, 95, 96, 97, 98, or 99% of the proteins of the original keratin material before fractional precipitation took place. For example, the fractional keratin precipitation method can remove between 5% and 80% of the proteins in the original keratin material, meaning that the precipitated keratin fraction can comprise 20% to 95% of the original keratin. More preferably, the precipitation method can remove between 5% and 20% of the proteins that are contributing to stiffness in order to afford a film with superior flexibility, therefore the precipitated keratin fraction can comprise 80% to 95% of the original keratin. Most preferably, the precipitation method can remove between 8% and 12% of the proteins that are contributing to stiffness in order to afford a film with superior flexibility, therefore the precipitated keratin fraction can comprise 88% to 92% of the original keratin.

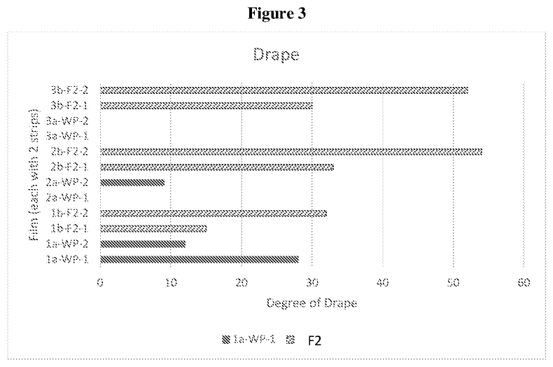

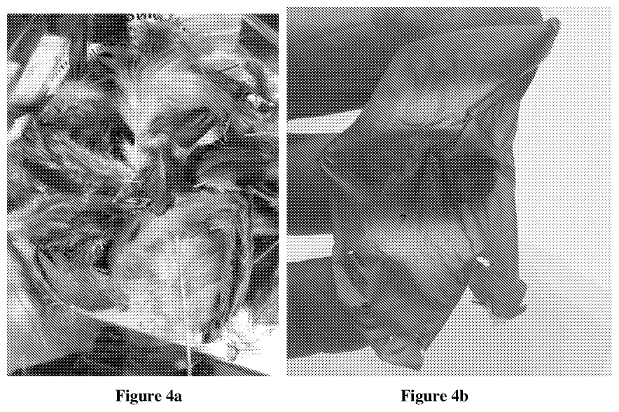

A film made from keratin subjected to the above fractional precipitation in comparison to a film made from the same keratin not subjected to the above fractional precipitation exhibits substantially greater drape angle. As described above, in a film with no drape, the drape angle would be zero degrees (0°) and with 100% drape the drape angle wound be ninety degrees (90°). When comparable sample thicknesses and similar conditions of ambient temperature and humidity during the time that the measurements are obtained, the film of the present invention has a drape angle of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82 82, 83, 84, 85, 86, 87, 88, 89, or 90° (or any amount in between) more than that of a film made from the same keratin not subjected to the above fractional precipitation. Examples can be seen in FIG. 3.

This method of film formation is amenable to the inclusion of additives that can be dissolved in the water used to create the slurry or insoluble particles that can be co-suspended with the keratin. Water soluble polymers that may impart beneficial properties include hydroactive, film forming polymers such as CMC, hydroxypropyl cellulose, pullulan, pluronics, alginate, polyethylene glycols, dextrans, and the like. Such polymers impart additional strength and/or water dispersibility. As mentioned above, wound dressing changes typically require cleansing or debridement of the wound including complete removal of previously applied dressings materials. Fragmentation of adherent keratin film prior to dressing change could make the dressing change more time-consuming, whereas a hydroactive additive facilitates moisture uptake that imparts dispersibility such that keratin residues can be simply rinsed off, as illustrated in Example 14.

The ability to blend insoluble substances with keratin particles in the slurry is especially advantageous as they can either be blended throughout or deposited separately to create a layered composite. Human amniotic membrane in powdered form is one such additive that would enable more efficient use of this natural membrane. Currently the size of an amniotic membrane wound dressing is limited by the size of the placenta from which it is procured and processed. Scraps that are produced during the cutting of the membranes to saleable sizes are generally wasted or processed into powder for use as an injectable substance that is mostly sold for unproven, dubious uses other than cutaneous wound healing. Reclaiming amniotic membrane scraps in the form of powder for use in wound healing is enabled by the present invention by means of blending amniotic membrane powder into the bespoke keratin slurry, as illustrated in Example 9. It can be readily appreciated that the amniotic membrane-containing keratin film wound dressing of the present invention has no limitation regarding its size and shape.

An especially useful concept also enabled by the above insoluble particle blending embodiment of the present invention is to obtain such particulate additives from skin epidermis or conical epithelium because of the naturally antimicrobial properties of these parts of the body that interface with the external environment, as discussed previously. Porcine sources for these tissues are preferred for reasons of cost, availability, and the precedent of porcine-sourced biomaterials having been safely used in human clinical products for many decades. The use of porcine epidermis is illustrated in Example 10 and the use of porcine corneal epithelium is illustrated in Example 11.

Since conical epithelium is relatively less available than skin, an efficient use of this additive is to apply it to the wound-contacting surface of the dressing rather than dispersing it throughout the film. Surprisingly, it was discovered that particles of decellularized porcine corneal epithelium self-adhere upon drying to produce a very thin, yet self-supporting membrane. As illustrated in Example 11, a bilayer structure was created by simply pouring a slurry of the fractioned keratin over a previously made corneal epithelium membrane. The layers of the resultant composite were intimately bonded together and exhibited greater clarity in comparison to the portion of the film that was not bonded to corneal epithelial membrane.

Also disclosed are methods of treating a subject in need thereof by providing the film disclosed herein. The film can be applied to any area in need of treatment, including but not limited to wounds, ulcers, burns, incisions, and major traumatic injuries as well as minor cuts or abrasions. The films of the present invention because of their softness and drape are especially desirable in covering deep, irregular wounds such as diabetic foot ulcers. An example of a medical need of critical importance is treatment of massive burn wounds that have previously been infected and must heal without reinfection to enable patient survival. The film disclosed herein featuring corneal epithelial tissue on its wound-contacting surface can address that need Other such situations in which these films will be of value will be readily apparent to experienced medical and surgical practitioners.

### EXAMPLES

Embodiments are described by the following examples, which are provided for illustration, not limitation.

### Example 1. Fractional and Non-Fractional Precipitation of HPE

Hair protein extract (HPE) was obtained using the Shindai process as described in U.S. Pat. No. 9,072,818. Briefly, human hair clippings were collected from approved hair salons, washed with shampoo, rinsed, dried, and exhaustively extracted with a mixture of chloroform and methanol using a Soxhlet extractor to remove lipids. The clean, delipidized hair was then soaked for 3 days at 50° C. in a pH 8.5 solution of 0.1N tris-buffer containing 5M urea, 2.5M thiourea, and 5% BME. The ratio of hair to solution was about 10 grams of hair per 100 mL of solution. Clear HPE solution was obtained by removal of the undissolved gelatinous residue by passing first through a strainer and then through filter paper.

Approximately 400 mL of filtered HPE were placed in a 2-liter glass beaker. A motor-driven rotor-stator homogenizer was lowered into the HPE solution and vigorous mixing commenced while slowly adding a total 1,200 mL of ethyl alcohol. The resultant precipitate was collected in a fine-mesh filtration sock and discarded.

The ethanolic solution of HPE resulting from removal of precipitate was then subjected to further precipitation by placing in a large glass bottle, adding an equal volume of acetone, securely capping the bottle, and vigorously mixing the liquids by shaking the bottle, after which, upon standing, precipitation gradually occurred. This precipitate was collected on analytical-grade filter paper, taking care not to let the precipitate dry. Once filtration was complete the paper was removed from the filter funnel and carefully folded in half and placed on several layers of paper towels. The precipitate was then blotted to remove most of the fluids by compressing with additional layers of paper towels, taking care not to extrude precipitate beyond the edges of the filter paper. The precipitate was collected upon unfolding the filter paper, placed into a jar, and tightly sealed with a screw-cap lid. This material is now referred to as “fractional precipitate” or “F2” or in some instances as “Gen-2”.

A sample of non-fractionally precipitated HPE was obtain by a similar process in which an organic solvent was slowly added to HPE solution with vigorous mixing by means of a homogenizer, except that acetone only was added to the HPE solution. The resultant precipitate was collected directly on analytical filter paper, blotted to remove excess liquids, and stored in a tightly sealed jar. This material is now referred to as “whole precipitate” or “WP”.

### Example 2. Conversion of Fractionally Precipitated and Non-Fractionally Precipitated HPE into Films

A mold for use in casting the film was made by cutting an 8-centimeter diameter hole in a ¼ inch thick sheet of silicone rubber and laminating this onto a second, solid sheet of silicone rubber of the same dimensions. The lamination was made temporary by simply coating the sheets to be contacted to each other with a thin layer of petrolatum to ensure a water-proof bond.

The damp WP and F2 precipitates of Example 1 were separately weighted, separately combined with distilled water, and thoroughly mixed with a magnetic stir bar on a magnetic stir plate until homogeneous slurries were obtained. Typically, magnetic mixing alone is usually not enough to disperse all lumps of precipitate, in which case a motor-driven homogenizer was subsequently used to create a smooth suspension, with care taken not to generate any foam.

The ratio of damp precipitate to water was 30 grams of precipitate to 135 mL of water. Each 3-gram precipitate slurry in water was used to make one 8-cm diameter film by pouring the slurry into the silicone rubber mold and allowing the water to evaporate completely. Removal of the cured keratin film from the mold was facilitated by removing the top layer of silicone from the bottom layer, flexing the keratin-coated bottom layer of silicone, gently picking up an edge of the keratin film disc and peeling it off the silicone rubber surface.

### Example 3. Proteomic Analysis of Fractionated Keratin Film Vs. Non-Fractionated Keratin Film

Samples of the WP and F2 films as made in Example 2 were submitted to the Systems Mass Spectrometry Core Facility at the Georgia Institute of Technology, Atlanta, Ga., for proteomic analysis. Significant differences in the profiles of keratin subtypes were detected and quantified, as presented in FIG. 1.

### Example 4. Determination of the Flexibility of Fractionated Keratin Film Vs. Non-Fractionated Keratin Film

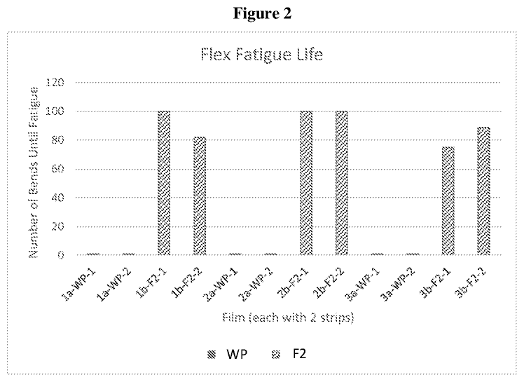

Samples of WP and F2 films made in Example 2 were cut into one-centimeter wide strips and the thicknesses measured at regular intervals across the length of each strip and averaged together to report an average thickness. The film thicknesses, flex-fatigue life data, and drape angle measurements are listed in Table 1 below and presented in FIG. 2 (flex-fatigue life) and FIG. 3 (drape angles). Note that in samples with comparable thickness (i.e. 1a & 1b vs. 2a & 2b) the overall flexibility of fractionated F2 films was dramatically greater than the flexibility of non-fractionated WP films, which in some cases exhibit no drape and broke upon being subjected to a single bending cycle.

TABLE 1

| Film | | Length | Thickness | Avg Thickness | | |
|---|---|---|---|---|---|---|
| (WP) | Strip | (cm) | (mm) | (mm) | FFL | Drape |
| 1a | 1 | 5 | 0.07 | 0.073 | 1 | 28° |
| (3 g WP | | | 0.07 | | | |
| slurry) | | | 0.08 | | | |
| | 2 | 5.2 | 0.09 | 0.077 | 1 | 12° |
| | | | 0.07 | | | |
| | | | 0.07 | | | |
| 2a | 1 | 3.2 | 0.13 | 0.113 | 1 | 0° |
| (6 g WP | | | 0.09 | | | |
| slurry) | | | 0.12 | | | |
| | 2 | 4.5 | 0.1 | 0.100 | 1 | 9° |
| | | | 0.1 | | | |
| | | | 0.1 | | | |
| 3a | 1 | 3.4 | 0.16 | 0.187 | 1 | 0° |
| (9 g WP | | | 0.23 | | | |
| slurry) | | | 0.17 | | | |
| | 2 | 3.7 | 0.15 | 0.153 | 1 | 0° |
| | | | 0.16 | | | |
| | | | 0.15 | | | |
| 1b | 1 | 3.2 | 0.07 | 0.066666667 | >100 | 15° |
| (1 g F2 | | | 0.06 | | | |
| slurry) | | | 0.07 | | | |
| | 2 | 4.6 | 0.11 | 0.086666667 | 82 | 32° |
| | | | 0.09 | | | |
| | | | 0.06 | | | |
| 2b | 1 | 5.2 | 0.1 | 0.096666667 | >100 | 33° |
| (2 g F2 | | | 0.1 | | | |
| slurry) | | | 0.09 | | | |
| | 2 | 4.5 | 0.08 | 0.09 | >100 | 54° |
| | | | 0.11 | | | |
| | | | 0.08 | | | |
| 3b | 1 | 3.9 | 0.13 | 0.14 | 75 | 30° |
| (3 g F2 | | | 0.15 | | | |
| slurry) | | | 0.14 | | | |
| | 2 | 5.2 | 0.14 | 0.133333333 | 89 | 52° |
| | | | 0.13 | | | |
| | | | 0.13 | | | |

WP = Whole Precipitate
F2 = Fractionated

### Example 5. Fractional and Non-Fractional Precipitation of Feather Keratin Protein Extract (FPE), Formation of FPE Films, and Comparison of Film Flexibility

A package of non-dyed, naturally colorful, clean bird feathers was purchased from a craft supply company and used as received by packing approximately 10 grams of feathers into each of four 4-ounce glass, screw-cap jars. To each jar was added approximately 100 mL of the same Shindai solution described in Example 1. Wooden tongue depressors were used to compress the feathers toward the bottom of the jars as the liquid was added, thereby enabling each jar to be filled to maximum capacity. The jars were tightly capped and placed in a 50° C. water bath for 3 days, whereupon the feathers mostly dissolved to become a mixture of dark solution and gelatinous residue that included fragments of the feather quills. FPE keratin solution was obtained from the mixture by filtration first through a steel strainer and then through analytical filter paper. The clear FPE solution was divided into two portions, one for fractional precipitation and the other for non-fractional precipitation, which was performed as described in Example 1 to yield damp precipitates that were identified again as “WP” (whole precipitate) for the non-fractionated precipitate and as “F2” for the fractionated precipitate. Films made by casting water slurries of the two materials were cut into 1-centimeter wide strips and tested for FFL and drape with the following results:

Drape Angle

The WP film did not drape at all.

The F2 film draped at a 58° angle.

Flex Fatigue Life:

The WP film snapped upon bending to 90° (FFL=<1).

The F2 film showed its first tear after 66 bending cycles (FFL=66).

### Example 6. Fractional Extraction of Dried ProgenaMatrix™ Granules

Surplus scraps of ProgenaMatrix™ were dried in air for several days until brittle and then ground with an electric coffee bean grinder and sieved to <300 microns. Although the Shindai process involves extraction of hair with a 5% by volume solution of BME in 5M urea and 2.5M thiourea, it was determined that dilution by half is sufficient to extract proteins from ProgenaMatrix™ granules.

A 0.10N solution of pH 8.5 tris buffer was prepared by dissolving 6.28 grams of tris acid (Sigma-Aldrich product no. RDD009) and 4.84 grams of tris base (Sigma-Aldrich product no. RDD0089) in 800 mL of deionized water. A stock solution of eluent was prepared by placing 150.0 grams of urea (Sigma-Aldrich product no. U5128) and 90.0 grams of thiourea (Sigma-Aldrich product no. T7875) in a 1,000 mL volumetric flask and filling half full with the tris buffer and warming the flask in hot water with periodic swirling until the solids dissolved. Additional buffer was then added to bring the volume up to the mark to yield 1 liter of a pH 8.5 solution of 0.10N tris, 2.5M urea, and 1.2M thiourea.

Four 100 mL volumetric flasks labeled numbers 1 through 4 were partially filled with the above stock solution. Using a precision pipetting instrument with disposable pipette tips, BME (Sigma-Aldrich product no. M6250) was added to each flask; 0.25 mL to flask no. 1, 0.50 mL to flask no. 2, 0.75 mL to flask no. 3, and 1.00 mL to flask no. 4. Each flask was then filled to the 100 mL mark with additional stock solution.

5.0 grams of the dried ProgenaMatrix™ granules were placed in a 600 mL beaker and about 150 mL of the stock eluent solution added. The mixture was stirred with a spin bar on a magnetic mixer for about one hour, during which time the granules hydrated and increased in volume. The mixture was then poured into a funnel lined with filter paper suspended over a beaker and the liquid allowed to drip into the beaker, labeled “extract 1”. The granules were then transferred back to the original 600 ml beaker and 100 mL of eluent solution no. 1 (0.25% BME) added and stirring continued for 30 minutes. The mixture was poured into another funnel lined with filter paper and the filtrate collected in another separate beaker, labeled “extract 2. The granules were extracted next with eluent solution no. 2 (0.50% BME) to obtain “extract 3”. Then with eluent solution no. 3 (0.75% BME) to obtain “extract 4”. Prior to collecting extract 5 the filter paper was accurately weighed, and the tare weight recorded. The granules collected in the filter paper, designated as “residue”, were kept in the filter paper, tied with cotton string, and allowed to dry.

Each of the extracts were then poured into separate beakers each containing 300 mL of denatured ethyl alcohol with stirring, which produced varying amounts of turbidity or precipitation, except for extract 1, which remained clear because it was obtained with stock eluent solution containing no BME. Each precipitate was then collected on weighted filter paper, the paper tied-up with cotton string, and allowed to dry. The residual buffer salts and urea and thiourea were washed out of the papers by placing the bundle of filters in a beaker and allowing tap water to run into the beaker and slowly overflow for about an hour. The filters were then rinsed with distilled water, blotted dry with paper towels, strings removed, and dried to constant weights in a food dehydrator at 95° F.

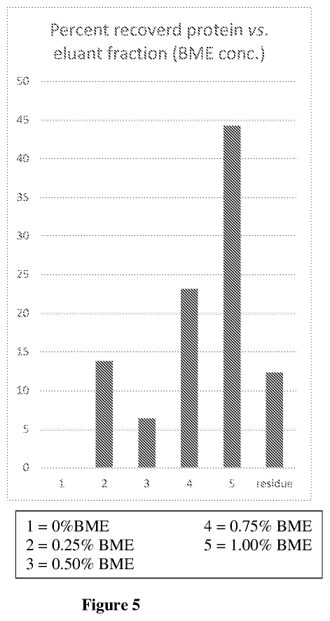

As shown in FIG. 5, increasing the BME concentration caused increased extraction of keratin, except in the case of the 0.50% BME eluent, which surprisingly extracted less keratin than both higher and lower BME concentration eluents.

### Example 7. Scale-Up of Fraction No. 2 for Use in Making a Wound Dressing

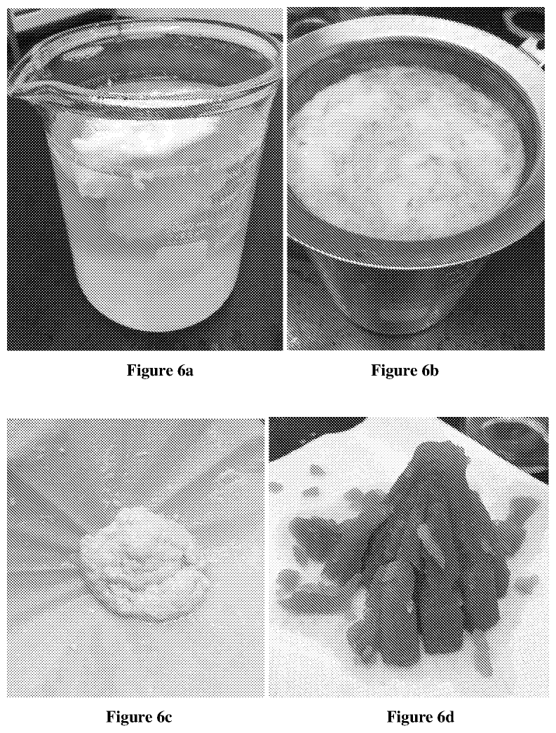

Ground particles of dried ProgenaMatrix™ (40.0 grams) were placed in a beaker with 400 mL of eluent stock solution as described in Example 6. and stirred with a spin bar on a magnetic mixer for about 30 minutes. The hydrated and swollen particles were collected on a filter and the filtrate discarded. The granules were returned to the beaker and another 400 mL of eluent stock solution containing 0.25% BME were added and stirred for about 30 minutes. The granules were again collected on a filter and the filtrate precipitated in 1,000 mL of denatured ethyl alcohol. At first the precipitate appeared merely as a turbid suspension, but soon agglomerated into a fluffy precipitate as seen in FIG. 6a. The precipitate was first collected on a strainer (FIG. 6b) and then further collected on filter paper. The recovered precipitate collected on filter paper (FIG. 6c) was dramatically less colored than the residual swollen granules of extracted ProgenaMatrix™ that were also collected on filter paper (FIG. 6d).

To purify fraction no. 2 and remove residual urea, thiourea, and buffer salts, the precipitate was stirred with a mixture of 300 mL deionized water and 300 mL of ethyl alcohol for about 30 minutes and then collected again on filter paper. When the precipitate was mixed with water only, the precipitate formed a slurry of extremely fine particles that clogged the filter paper. These fine particles were not formed when using the 1:1 water-ethanol purification solvent, however, which facilitated rapid recovery of the purified fraction no. 2 product. The collected precipitate, while still damp, was then placed in a tightly sealed screw cap jar and stored at 4° C. in a refrigerator.

### Example 8. Formation and Flexibility Determination of Keratin Film Made from Fractionally Extracted Vs. Non-Extracted ProgenaMatrix™

A slurry of the fractionally extracted precipitate of Example 7 was prepared by mixing with water and pouring into an 8 cm diameter silicone rubber mold made by combining two layers of ⅛″ thick silicone rubber, the top layer having a central hole of 8 cm in diameter. Enough slurry of the requisite percent solids was poured into the mold such that upon evaporation of the water and removal of the resultant film the weight of the film thus formed was approximately 25 milligrams per square centimeter (25 mg/cm²). A sample of ProgenaMatrix™ was removed from the package, rinsed with distilled water and allowed to dry in open air for several days alongside the above sample made from precipitated fractional extract. The fractional extract film remained soft and pliable whereas the dried ProgenaMatrix™, which also weighed about 25 mg/cm², was stiff and broke upon bending.

### Example 9. Formation of a Keratin Wound Dressing Film Containing Amniotic Membrane Powder

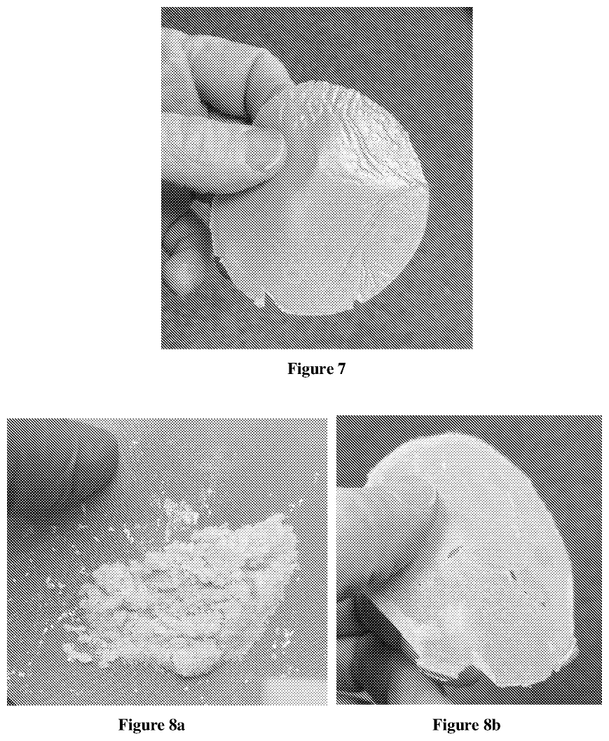

A 40-mg sample of dehydrated, powdered human amniotic membrane (AmnioFix®, MiMedx Group, Inc, Marietta, Ga.) was mixed with a slurry of the F2 precipitate of Example 1, cast on a silicone rubber mold, and the water allowed to evaporate as described in Example 2. The resultant film (shown in FIG. 7) contained approximately 10% by weight amniotic membrane and was soft and flexible.

### Example 10. Formation of a Keratin Wound Dressing Film Containing Particles of Porcine Epidermis

Pork skin purchased from a local market was trimmed to remove meat, flattened, and placed in a freezer until frozen solid. A hand-held grater was used to remove the epidermis and as little as possible of the underlying dermis by shredding it into particles of approximately 1-mm wide by 2-3 mm in length. The particles (2.16 grams) were placed in a 0.1% solution of SDS in distilled water in a 50 mL tube and the tube was gently rotated on a rotation device overnight at room temperature. The particles were separated from the SDS solution and rinsed with copious deionized water to remove all traces of the SDS, then rinsed with ethyl alcohol followed by acetone, and then allowed to dry in open air to yield 0.65 grams of dried particles. The dry particles were suspended in ethyl alcohol and comminuted by means of a motor-driven rotor-stator homogenizer. The skin particles were rather tough and did not yield a powder as desired. However, the particles were reduced from the original grated material down to a size range of about 0.2-1.0 mm in any dimension. The homogenized particles were collected on a filter (shown in FIG. 8a), mixed with a slurry of the F2 precipitate of Example 1, cast on a silicone rubber mold, and the water allowed to evaporate as described in Example 2. The resultant film (shown in FIG. 8b) contained approximately 10% by weight porcine epidermis particles and was soft and flexible.

### Example 11. Formation of a Keratin Wound Dressing Film Containing a Membrane of Porcine Corneal Epithelium

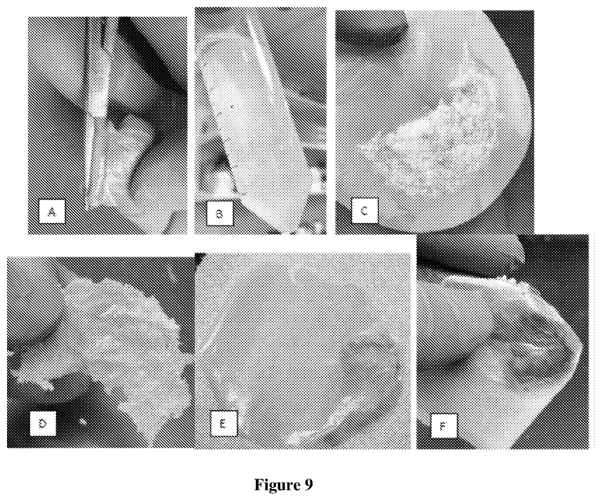

Frozen porcine corneas were purchased from Pel-Freez Biologicals, Rogers, Ark., and allowed to thaw. Four corneas were placed in a 70% solution of ethyl alcohol in water containing 5% sodium chloride in a 50 mL tube. The tube was gently rotated on a rotation device overnight at room temperature and then the corneas were removed and rinsed thoroughly with distilled water. The corneal epithelium was separated from the conical stroma of each cornea by pulling it off with the use of a hemostatic clamp as shown in FIG. 9a. The stromal tissues were discarded, and the epithelial tissues were placed in a 0.1% solution of SDS in distilled water in a 50 mL tube and the tube gently rotated on a rotation device for 3 days at room temperature. The tissues were then rinsed with copious deionized water and placed in a tube of ethyl alcohol and homogenized with the motor-driven rotor-stator homogenizer to create a suspension of finely minced tissue (FIG. 9b). The tissue particles were collected on a 80-micron nylon net filter (FIG. 9c) and allowed to dry in air, whereupon the particles self-adhered to become a film that was readily detached from the nylon (FIG. 9d). This specimen of film was placed on the silicone rubber mold of Example 2 and a slurry of F2 keratin as described in Example 1 was poured on top of the corneal tissue film and allowed to dry, which resulted in the formation of a soft and flexible film. The portion of the film that was a bilayer composite of human hair keratin and decellularized porcine corneal epithelium tissue (FIG. 9e) was determined by weight to be 70% fractionated HPE keratin and 30% decellularized porcine corneal epithelial tissue. Surprisingly, as shown in FIG. 9f, the presence of the conical epithelium imparted enhanced clarity and transparency to the composite film, which was equally soft and flexible in comparison to the portion of the film that did not contain corneal epithelium film.

### Example 12. Formation of a Keratin Wound Dressing Film Containing Pullulan

Pullulan, a polysaccharide polymer, was purchased from TCI America, Portland, Oreg. (Tokyo Chemical Industry Co., Ltd.), and dissolved in deionized water and combined with the damp F2 precipitate of Example 1 at the ratio of 8 grams of pullulan to 33 grams of F2 precipitate to 150 mL of water. These ingredients were thoroughly blended together using a tissue grinder to obtain a homogeneous slurry. Approximately 18 mL of the resultant slurry was poured into the silicone rubber mold of Example 2 and allowed to dry completely. The resultant film was soft and flexible.

### Example 13. Formation of a Keratin Wound Dressing Film Containing Pluronic-F127

Pluronic® F-127, a copolymer surfactant, was purchased from Millipore-Sigma Chemical Co., St. Louis, Mo. (product no. P2443) and used to create a film with the F2 precipitate of Example 1 using the same procedure of Example 12, except that pluronic was used as a substitute for pullulan. The resultant film was soft and flexible.

### Example 14. Determination of the Dispersibility of a Keratin Wound Dressing Film Containing Pullulan

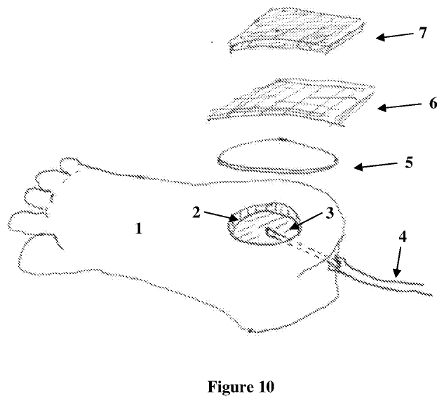

A medical model of a human foot made of silicone rubber featuring an oval shaped (3.6 cm long by 2.8 cm wide) diabetic foot ulcer on its plantar surface comprising an approximately 4.5 cm³ cavity having an irregular surface was purchased from Health Edco, Waco, Tex. The cavity was used to simulate an exuding wound by means of pumping artificial wound fluid into the ulcer cavity via a tube attached to a hypodermic needle that was inserted as shown in FIG. 10.

An artificial wound fluid was prepared by dissolving 1.47 g (10 mM) calcium chloride (CaCl₂ 2H₂O), 11.7 g (200 mM) sodium chloride (NaCl), 4.85 g (40 mM) tris(hydroxymethyl)aminomethane, and 20.0 g bovine serum albumin (BSA, protease free) in deionized water, adjusting the pH to 7.4 with dilute hydrochloric acid, placing the solution in a one-liter volumetric flask and adding additional deionized water to the one-liter mark.

Preparation for the test was made by draping the film of Example 12 into the cavity such that it was in intimate contact with the floor and sides of the cavity and reflected at its edges to be flush with the surrounding surface of the model. Several commercially available dressing materials commonly used in clinical practice were then applied as shown in FIG. 10. The first dressing material applied on top of the keratin-pullulan film was Adaptic® non-adhering dressing (Systagenix, Acelity/KCL, San Antonio, Tex.) made of knitted cellulose acetate fabric and impregnated with a petrolatum emulsion. Next were placed two layers of gauze pad (Band-Aid® brand gauze, Johnson & Johnson), and finally the foot was wrapped with a compressive wrap taken from a box of Coban™2, (3M, St. Paul, Minn.).

The test was performed by using a syringe pump to instill artificial wound fluid under the test material. This fluid absorbed into and transpired through the test material and subsequently wicked into and was absorbed by the overlying layers of dressing materials. The syringe pump delivered fluid at the rate of 100 microliters per hour, which was estimated to be the rate of wound fluid exudation of a moderately exuding wound of this size in a typical human patient suffering from a non-healing wound of the foot at this location. Since wound dressings in clinical practice in an outpatient setting are typically changed on a weekly basis, the test was run for 7 days, during which time fluid was continually infused into the cavity underneath the test material.

At the conclusion of the 7-day period, dressings were removed to reveal the condition of the test material. It was observed that the film of Example 12 under these test conditions remained contiguous with the cavity surfaces but had completely disintegrated. Thus, the residue of the film was readily removed as would be required during dressing changes by gently wiping the hydrated residue off with a moistened piece of gauze.

### Example 15. Evaluation of a Keratin-Pullulan Wound Dressing Film in the Healing of Delayed-Healing Wounds in a Diabetic Mouse Model

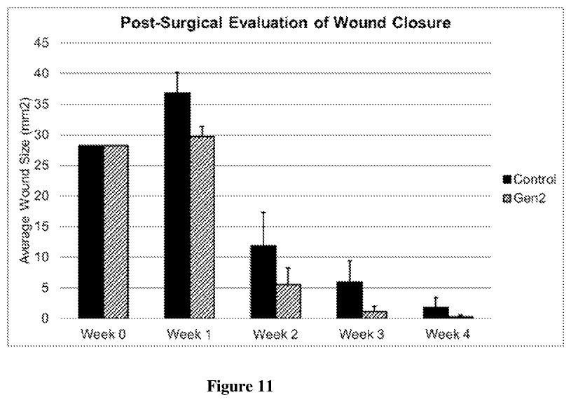

The wound healing efficacy of the wound dressing film of Example 12 was determined in an obese diabetic mouse model of delayed wound healing of full-thickness excisional wounds as described by Ingraham, et al. Briefly, eight leptin-receptor deficient (db/db) obese mice received four equal 6 mm diameter full thickness excisional wounds made by pressing a 6 mm biopsy punch through a fold of skin into a wooden tongue depressor to created pairs of wounds. The film of Example 12 was cut into discs of 10 mm diameter, sterilized with ethylene oxide gas, and applied directly over two of the wounds on each of 8 mice with the other two wounds on each mouse serving as untreated controls. All wounds, whether designated as untreated control or not, were dressed (on top of test material) with Iodosorb® (Smith & Nephew), Skintegrity® (Medline), Band-Aid® Gauze (J & J) and covered with Tegaderm™ transparent film dressing (3M). Dressings and test materials were changed weekly (as is done clinically) and the surface area of each residual open wound measured (mm²). As shown in FIG. 11, wounds treated with the wound dressing film of Example 12 healed at a faster rate than wounds treated without the F2 keratin film.

While the invention has been illustrated by a description of various embodiments and while these embodiments have been described in considerable detail, it is not the intention of the applicant to restrict or in any way limit the scope of the appended claims to such detail. Additional advantages and modifications will readily appear to those skilled in the art. Thus, the invention in its broader aspects is therefore not limited to the specific details, representative apparatus and method, and illustrative examples shown and described. Accordingly, departures may be made from such details without departing from the spirit or scope of applicants' general inventive concept.

### REFERENCES

### Patent References

Barrows T H, “Biomaterials made from human hair”, U.S. Pat. No. 9,072,818, Jul. 7, 2015
Kelly, et al., “Wound care products containing keratin”, U.S. Pat. No. 7,732,574, Jun. 8, 2010
Van Dyke M E, and Teli T, “Keratin compositions for treatment of bone deficiency or injury”, U.S. Pat. No. 9,220,754, Dec. 29, 2015
Van Dyke, et al., “Keratin nanomaterials and methods of production”, US Patent Application Publication 2017/0051027, Feb. 23, 2017

### Additional References

Brunner H and Brunner A, “Fractionation of tyrosine-rich proteins from oxidized wool by ion-exchange chromatography and preparative electrophoresis, Eur J Biochem, 32, 350-355 (1973)
de Guzman R, et al, “Mechanical and biological properties of keratose biomaterials”, Biomaterials 32 (2011) 8205-8217
Dealey C, et al., “A study comparing two objective methods of quantifying the production of wound exudate”, J Wound Care, 15(4), 149-153 (2006)
Frykberg R G and Banks J, “Challenges in the treatment of chronic wounds”, Advances in Wound Care, 4(9), 560-582 (2015)
Fujii T and Ide Y, “Preparation of translucent and flexible human hair protein films and their properties”, Bio Pharm Bull, 27(9), 1433-1436 (2004)
Fujii T, “Hair keratin film as a substitute device for human hair”, J Biol Macromol, 12(1), 3-15 (2012)
Fujii T, et al., “Convenient procedures for human hair protein films and properties of alkaline phosphatase incorporated in the film”, Bio Pharm Bull, 27(1), 89-93 (2004)
Ingraham C, et al., “Keratin Hydrogel Film Accelerates Wound Closure in a Diabetic Mouse Model of Delayed Wound Healing”, Annual Hilton Head Workshop: Discovery, Technologies and Translation, Sea Pines Plantation, Hilton Head, S.C., May 13-16, 2015
Levesque M, et al., “Skin wound healing in axolotls: A scarless process”, J Exp Zool (Mol Dev Evol) 314B, 684-697 (2010)
Ma B, et al., “Pure keratin membrane and fibers from chicken feather”, Int J Biol Macromol, 89, 614-621 (2016)
Mir M, et al., “Synthetic polymeric biomaterials for wound healing: a review”, Progress in Biomaterials, 7, 1-21 (2018)
Momeni M, et al., “In vitro and in vivo investigation of a novel amniotic-based chitosan dressing for wound healing, Wound Rep Reg, 26, 87-101 (2018)
Muhammad A, et al., “In Vitro Wound Healing Potential and Identification of Bioactive Compounds from Moringa oleifera Lam”, BioMed Research International, Volume 2013 (2013), Article ID 974580, 10 pages http://dx.doi.org/10.1155/2013/974580
Poranki D, et al., “Evaluation of skin regeneration after burns in vivo and rescue of cells after thermal stress in vitro following treatment with a keratin biomaterial”, J Biomat Appl, 29(1), 26-35 (2013)
Raymond S, et al., “Delayed healing associated with silver sulfadiazine use for partial thickness scald burns in children”, The American Surgeon, 84, 836-840 (2018)
Reichl S, et al., “Keratin films for ocular surface reconstruction”, Biomaterials, 32, 337-3386 (2011)
Richter J R, et al., “Structure-property relationships of meta-kerateine biomaterials derived from human hair”, Acta Biomater, 8, 274-281 (2012)
San Emeterio C, et al., “Human keratin-based materials promote a pro-regenerative immune environment in skin injury”, 2018 Regenerative Medicine Workshop at Charleston: Synergizing Science, Engineering, and Clinical Translation, Wild Dunes Resort, Isle of Palms, S.C., Mar. 21-24, 2018
Tam C, et al., “Cytokeratins mediate epithelial innate defense through their antimicrobial properties”, J Clin Invest, 122(10), 3665-3677 (2012)
Vasconcelos A and Cavaco-Paulo A, “The use of keratin in biomedical applications”, Current Drug Targets, 14, 612-619 (2013)
Waters M, et al., “Keratin biomaterials augment anti-inflammatory macrophage phenotype in vitro”, Acta Biomater, 66, 213-223 (2018)
Zasloff M, “Defending the cornea with antibacterial fragments of keratin”, J Clin Invest, 122(10), 3471-3473 (2012)

1. A film comprising a fraction of keratin proteins separated from an original extracted keratin protein mixture, wherein said film comprises consolidated particles of said fraction; wherein said film possesses a greater flex-fatigue life or exhibits a greater drape angle than a comparable film comprising consolidated particles of the original extracted keratin protein mixture from which it was fractionally separated.

2. The film of claim 1 in which said original extracted keratin protein mixture is obtained from human hair.

3. The film of claim 1 in which said extracted keratin proteins are obtained from feathers.

4. A process of obtaining a fraction of keratin proteins, the process comprising:
a. Providing a solution of keratin proteins by extracting a keratin raw material in an alkaline aqueous solution comprised of a reducing agent, a denaturing agent, and buffer salts;
b. Fractionally precipitating keratins from the solution of keratin proteins of step a. by addition of a first water-miscible organic solvent and collecting a solution of the remaining non-precipitated keratins by filtration;
c. Further fractionally precipitating keratins from the solution of step b. by addition of a second water-miscible organic solvent and collecting the precipitated fraction of keratin solids on a filter; thereby obtaining said fraction of keratin proteins.

5. The process of claim 4, wherein said first water-miscible organic solvent is ethanol and said second water-miscible organic solvent is acetone.

6. A process of obtaining a fraction of keratin proteins, the process comprising:
a. Providing a keratin material in granular form;
b. Sequentially extracting keratin granules of step a. with a series of eluents having a constant concentration of denaturing agent and pH, but containing an increasing concentration of reducing agent;
c. Separately precipitating keratin content of each eluent in a water-miscible organic solvent and collecting each precipitate on a filter;
d. Selecting a precipitate fraction possessing a greater flex-fatigue life or exhibiting a greater drape angle than a comparable film comprising consolidated particles of the original extracted keratin protein mixture from which it was separated; thereby obtaining a fraction of keratin proteins.

7. The process of claim 6 wherein said keratin material of step a is a recycled keratin material.

8. A process of obtaining a film, wherein said processing comprises the steps of:
a. Providing a fraction of keratin proteins comprising precipitated keratin according to the process of claim 4;
b. Adding water to the precipitated keratin of step a and mixing to produce a slurry of particles;
c. Placing the slurry of step b into a mold;
d. Allowing the water to evaporate;
e. Removing the resultant film from the mold.

9. A film produced by the process of claim 8.

10. A film of claim 1 further comprising substances beneficial to wound healing selected from the group comprising human and animal tissues and membranes and substances derived from said tissues and membranes.

11. A film of claim 10 wherein said added substances form a layer on one side of the film.

12. A film of claim 10 in which said tissues are selected from the group comprising amniotic membrane, epidermis, and corneal epithelium.

13. A film of claim 1 further comprising a hydroactive polymer.

14. A film of claim 13 in which said hydroactive polymer is selected from the group comprising pullulan, pluronic, CMC, alginate, and dextran.

15. The film of claim 1, wherein the film comprising a fraction of keratin proteins separated from an original extracted keratin protein mixture can withstand at least 2 cycles more of flexing compared to the original extracted keratin protein mixture from which it is extracted.

16. The film of claim 1, wherein the film comprising a fraction of keratin proteins separated from an original extracted keratin protein mixture comprises 10° or greater drape angle compared to the original extracted keratin protein mixture from which it is extracted.

17. The film of claim 1, wherein the fraction of keratin proteins separated from the original extracted keratin protein mixture comprises 80% to 90% of the proteins in the originally extracted keratin material.

18. A method of treating a subject, the method comprising administering the film of claim 1 to a subject in need thereof.

19. The film of claim 1, wherein said fraction of keratin protein comprises a lower content of keratins KRT85 and KRT86 relative to said original extracted keratin protein mixture.
